(19) United States
(12) Patent Application Publication
(10) Pub. No.: US 2021/0132049 A1
(43) Pub. Date: May 6, 2021

(54) Simultaneous Parallel Signal Amplification and Analyte-Ligand Capture Functions

(71) Applicant: George Mason University, Fairfax, VA (US)

(72) Inventors: Lance A. Liotta, Manassas, VA (US); Alessandra Luchini Kunkel, Manassas, VA (US); Marissa A. Howard, Fairfax, VA (US); Sameen Yusuf, Fairfax, VA (US); Sara Sharif, Fairfax, VA (US); Rohit L. Madhu, Fairfax, VA (US)

(21) Appl. No.: 16/617,374

(22) PCT Filed: May 29, 2018
(86) PCT No.: PCT/US2018/034955

§ 371 Date: Nov. 26, 2019

Related U.S. Application Data
(60) Provisional application No. 62/511,474, filed on May 26, 2017.

(51) Int. Cl. G01N 33/545; G01N 33/543; G01N 33/569; B01D 15/38; G01N 27/327

(52) U.S. Cl. CPC G01N 33/545; G01N 33/5438; G01N 27/3273; B01D 15/3809; G01N 33/5695

(57) ABSTRACT

A device and system for conducting accurate diagnosis of states of disease or health, including cancer, with high sensitivity and specificity is described. The system employs a portable cassette-based testing system which is configured to detect pp analytes captured by hydrogel particles equipped with affinity bait(s) and an amplification reporter system. The affinity baits bind to a wide range of analytes, including: metabolites, proteins, nucleic acids, lipids, hormones, cytokines, growth factors, biomarkers, virus particles, exosomes, bacteria, fungi, drug compounds, synthetic organic compounds, volatile odorants, toxicants, and pollutants. The affinity baits bind with extremely high affinity, preserving the captured analyte. This system can increase the sensitivity of detection of said analytes up to ten-thousand-fold.

### CONTINUITY

This application is a non-provisional patent application of provisional patent application No. 62/511,474, filed on May 26, 2017, and priority is claimed thereto.

### FIELD OF THE PRESENT INVENTION

The present invention relates to diagnostic medical testing, and more specifically relates to a rapid diagnostic test for the detection of diseases, cancers, and monitoring effects of therapy.

### BACKGROUND OF THE PRESENT INVENTION

Roughly a third of the world's population is infected with tuberculosis (TB). In America, the TB incidence burden is much lower, however to fully eliminate its incidence in the US there needs to be a gold standard method of identifying those with latent tuberculosis (LTBI). LTBI is an inactive form of the TB disease. The diagnostic tests available to individuals who are at risk for LTBI and TB infections are indirect (quantification of immunological response), invasive (requiring blood sample or an injection), time consuming (24+ hours), and expensive. TB biomarkers demonstrate low concentrations in urine, and current urinary assays are neither sensitive nor rapid enough. We have developed an electrical, paper-based immunoassay using high affinity capture hydrogel particles and amperometric sensors to provide a sensitive, specific, and quantitative method for LTBI diagnosis. The hydrogel particles employed are an open mesh network of polymers with high affinity chemical baits dyed to their surface. They exhibit strong protein capturing abilities. Incorporating copper dyes onto the hydrogel particles' surface with enzymatic reactions within their internal volume enables their presence to be detected electrically. By coupling an Arduino Pro Mini microcontroller board with amperometric sensors, we have demonstrated that copper dyed hydrogel particles with enzymatic amplification built in, can be reliably detected by monitoring the current response of the hydrogel particles. The capability of measuring the hydrogel NP's presence allows us to create an electrical lateral flow immunoassay that can quantitatively detect TB biomarkers within a urine sample. It should be understood that an alternate microcontroller board aside from an Arduino may be employed, however an Arduino is selected as preferred due to cost and ease of use.

As such, there is an immense need to develop a biosensor system that provides sufficient signal while additionally detecting low abundance biomarkers. Current biosensor technology lacks the sensitivity to detect low abundance biomarkers and amplify the signal without additional equipment. This includes a need for faster and more accurate tests for a wide range of diseases and cancers. Thus, there is a need for a method for detection of an analyte-ligand binding pair system in an open-mesh cage particle with a separate built-in chemical amplification report system to allow for increased sensitivity. This technology advances the field of biosensors because it allows for detection of diseases, cancers, or monitoring of the body by implementing a system that both captures the target biomarker and amplifies a signal for analysis.

Tuberculosis (TB) is caused by the bacteria M. tuberculosis that has deleterious effects on the respiratory system and is an epidemic that affects more than 9.6 million people globally. One-third of the global population is infected with latent TB (L TBI). These individuals cannot spread the disease, and do not exhibit symptoms. LTBI patients have a 0-15% chance of developing active TB if they come into contact with another individual infected with active TB, or they may develop other infectious diseases such as HIV on their own. Antimicrobial treatment will no longer be effective once the latent TB infection has been activated. Thus, it is vital for L TBI patients to be diagnosed early and receive treatment immediately.

Current diagnostics on the market include: 1) Tuberculin Skin Test (TST), 2) Interferon Gamma Release Assay (IGRA), 3) GeneXpert, 4) Determine TB-LAM. Specifically, the TST and IGRA are the CDC's recommended diagnostic for identifying LTBI within patients. All of these TB diagnostics fail to produce inexpensive, non-invasive, and sensitive in a rapid and quantitative manner, Table 1. The ideal test would be noninvasive and require a sample of a bodily fluid such as urine. However, TB biomarkers are present at low concentrations in urine, and currently available urinary assays are neither sensitive nor rapid enough. Current urinary tests perform best for those demonstrating immunodeficiency with low CD4 cell counts (<50 cells/μL)^{[31]}. Since 15% of TB patients are co-infected with HIV, this means that urinary assays fail to detect TB for 85% of the 9.6 million TB patients worldwide.

According to the World Health Organization (WHO), there is an urgent need for a highly sensitive, rapid point of care diagnostic test for active TB and L TBI. The goal of the present invention is to develop an electrical, paper-based immunoassay using high affinity capture hydrogel particles to provide a sensitive, specific, and quantitative method for L TBI diagnostics from urine samples.

### Diagnostic Analysis Currently Available for Tuberculosis

The GeneXpert is a top of the line rapid Mycobacterium tuberculosis (MTB) and rifampicin (RIF) mutation rapid diagnostic tool. The Xpert was endorsed by the WHO in 2011 for its self-contained, automated molecular assay for rapid diagnosis. This technology was originally created to detect anthrax, but in recent years it has been altered to detect pulmonary TB. Utilizing sputum samples, the GeneXpert can detect up to 10-50 cfu/mL (colony forming units per milliliter) compared to regular smear microscopy methods of detection of 10,000 cfu/mL. This detection limit was demonstrated in laboratory settings, however it was unable to be reproduced in field testing. Although this technology lessens the need to culture TB, lowers the time of diagnosis, and has been better than conventional smear microscopy, the unit cost has been quoted at $17,000 USD. In addition, the machine needs user training and requires a continuous electrical supply, and controlled storage environment.

### Tuberculin Skin Test

The Mantoux tuberculin skin test is a visual test that consists of applying TB antigens (Specifically 0.1 mL off PPD RT23 which is a protein derivative of Mycobacterium tuberculosis) intradermally by a trained physician. Usually this test is done on the left forearm to minimize interpretation errors. The results are interpreted 48-72 hours later. Results are based upon the size of diameter of the reaction under the skin.

The positive result is standardized to induration sizes of 5 mm, 10 mm, or 15 mm. These different positive results could be the result of many factors, including other diseases, physiological characteristics, or even recent exposure to TB. For example, an HIV patient could have an induration size of 5 mm and have a positive result. The advantage of this test is that it is simple to administer, nevertheless it is a visual assessment where the interpretation is wholly based on the health professional's estimate. Some disadvantages include: the patient must not interfere with the site and come back within 48-72 hours. Moreover, this test lacks specificity and false negatives do occur, which could be attributed to a weak immune response, recent TB infection (8-10 weeks), infant physiology, or recent vaccine administration.

### Enzyme-Linked Immunosorbent Assay (ELISA)

Enzyme-Linked Immunosorbent Assay (ELISA) is a 96-well plate-based assay technique used to detect and quantify biological substance such as proteins, antibodies, peptides, and hormones. ELISA detects the analyte by measuring the conjugate enzymatic activity after incubation. The antibody-antigen conjugation creates a colorimetric response. The total amount of color produced is attributed to the amount of that substance present.

There are three main ELISA formats: Direct Assay, Indirect Assay, and the Sandwich Assay; each have their own advantages and disadvantages. The direct assay utilizes a primary antibody conjugate that is associated with an enzyme, the indirect assay has a secondary antibody conjugate that is binded to an enzyme, and the capture assay utilizes a capture antibody as well as a primary and secondary antibody conjugate with an enzyme link. Moreover, in all of these cases, a gold particle or a capture antibody is used to bind the antibody conjugates to the well.

Currently, the LAM-ELISA test is being utilized to study TB patients coinfected with advanced HIV and Tuberculosis, but a small sample size and inadequate evaluations of HIV patients with advanced immunosuppression limited the overall efficacy of the experiment. Thus, this project is mostly preliminary and depicts the need for more research in this intersection of TB and HIV.

### Interferon Gamma Release Assay (IGRA)

There are two FDA approved IGRA tests: QuantiFERON®-TB Gold In-Tube test and T-SPOT® TB Test. Unlike the GeneXpert, ELISA, and lateral flow tests, IGRA measures an individual's immunological response to the M. tuberculosis bacteria. The test is administered by collecting a fresh blood sample from the patient, and the blood sample is then mixed with TB antigens. When white blood cells come into contact with the TB antigens, they release interferon-gamma (IFN-g). Depending on the specific test, either the concentration of IFN-g or cells producing IFN-g is measured.

IGRA tests can be administered in place of the traditional skin test, however the CDC does not recommend using both tests simultaneously. Though IGRA cannot differentiate between LTBI and active TB, previous vaccination does not produce false positive results. Furthermore, multiple IGRA tests will not lead to false positive results since the test is administered ex-vivo, unlike the tuberculin skin test. Nevertheless, the test does take at least 24048 hours to process, is costly (QuantiFERON: $227.00; T-SPOT: $160.00), and requires a trained professional to interpret, transport, and administer the test.

### Determine TB-LAM

A lateral flow immunoassay test (or simply, a strip test) is an immunoassay designed to detect the present or absence of an analyte of interest from a sample without the use of complex and expensive instruments. A standard lateral flow test strip includes an absorbent pad where the sample being tested is applied, called the sample pad. There is also a conjugate or reagent pad where a combination of specific antibodies and latex microspheres or colloidal gold nanoparticles are location to capture the analyte of interest. The reaction membrane is usually either a nitrocellulose membrane or a cellulose acetate membrane. Anti-target analyte antibodies are applied across the membrane as the test (or capture) line; there is also a control line where specific conjugate antibodies are applied. Another absorbent pad is location at the end of the test to help the sample, through capillary action, completely flow across the reaction membrane to be collected at the end.

The existing lateral flow immunoassay that has emerged as a potential point-of-care (POC) test for Mycobacterium tuberculosis (MTB) is known as Determine TB-LAM according to the WHO. This lateral flow assay is designed to detect the antigen lipoarabinomannan (LAM) location within the cell wall of MTB, which is typically excreted in the urine. One of the advantages of this assay is that it examines urine as opposed to sputum samples, which is safer and easier to collect, especially in areas of limited resources. The Determine TB-LAM test kit only requires one visit to the clinic, costs as little as $3.50 per test, and produces results within 30 minutes. Furthermore, using Determine TB-LAM requires minimal training and no electric supply or intricate instruments.

Disadvantages of the assay include a very low sensitivity of approximately 28.2% in active MTB patients, and in contrast, approximately 66.7% sensitivity when the T-cell (CD4 cell) count was less than 50 cells/μL. Further studies indicate that the TB-LAM test demonstrated increased sensitivity in patients that were HIV positive with a T-cell count below 200 cells/μL. These studies suggest that it is possible the Determine TB-LAM could significantly enhance the diagnosis of MTB in patients with HIV and other major diseases. These studies also denounce that, due to the test's limited sensitivity, it is necessary for it to be used in conjunction with another diagnostic test, such as smear microscopy, to be able to obtain enough sensitivity to diagnose or rule out MTB. This way, the TB-LAM lateral flow test can further increase sensitivity, particularly in the HIV patients. However, a thorough field assessment is required to evaluate the diagnostic efficacy of the test as a reliable immunoassay for detecting active MTB in HIV patients and in areas with limited resources.

### Objective

As such, it is the intent of the present invention to provide a rapid screening test for LTBI that is as simple to use as the standard pregnancy test. The device must combine the advantages of current practices and methods on the market as described above.

The most important characteristic of the test of the present invention is its sensitivity, which must be at least 95% to compete with ELISA and IGRA tests. For the first prototype, the goal was to obtain an analytical sensitivity of 1 ng/mL protein detection. This value can be improved upon for later prototypes and with a longer time frame. Furthermore, according to active disease screening recommendations, diagnostic tests must have at least 80% specificity. Though 80% specificity is comparably low for the current practices on the market, it is important to consider the sensitivity-specificity trade-off—it is better to have a higher rate of false positives rather than higher rate of false negatives.

### SUMMARY OF THE PRESENT INVENTION

This Summary of the Invention is provided to introduce a selection of concepts in a simplified form that are further described below in the Detailed Description. This Summary is not intended to identify key features or essential features of the claimed subject matter, nor is it intended to be used to limit the scope of the claimed subject matter.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

The present invention is an electrical, paper-based immunoassay which uses unique hydrogel particles to support efforts to produce diagnostics that are rapid, affordable, and user-friendly.

The device and system of the present invention employs a new class of hydrogel particle nanotechnology (referenced as nanoparticles or hydrogel particles) to create a novel one-step self-working, enzymatically amplified visual lateral flow immunoassay that achieves a sensitivity one hundred to one thousand times higher than conventional assays. The technology will be used to create urinary direct antigen tests for Active Tuberculosis (TB) and newborn Chagas disease, applicable to low resource community clinics addressing urgent worldwide medical need. The test has been validated in well characterized retrospective clinical specimens. The present invention employs a new class of porous hydrogel particles that contain internal high affinity bait chemistries. The chemical baits, immobilized in the core of the hydrogel particles are a previously unexplored class of modified organic molecule dyes that bind pathogen glycans with extremely high affinity (Ko<=10·′3 M) and a very low off-rate.

There is a recognized need to create high affinity glycan ligands for diagnostics. We introduce here a new class of chemical affinity bait, a copper complex reactive dye Reactive Blue 221 (RB221). that binds and sequesters the TB carbohydrate glycan antigen LAM and the Chagas glycolipid antigen with extremely high affinity. When introduced into urine, the hydrogel particles containing RB221 harvest the glycan antigens with high efficiency while simultaneously dissociating interfering substances. in one step. in solution, in minutes. We have verified in infected patient samples that our affinity capture technology increases the sensitivity of LAM detection in the urine of patients with active TB by 100 fold or greater.

Solution phase enzyme amplification inside the hydrogel particle volume Visual lateral flow immunoassays have low sensitivity and cannot detect critical low abundance pathogen antigens in urine. Enzymatic amplification of immunodetection reactions is a common means of greatly increasing sensitivity. Nevertheless, enzymatic amplification requires sequential addition of reagents and washing steps to remove the unbound enzyme labeled antibody. In the past this has seriously prevented the creation of a one-step enzymatically amplified self-working visual lateral flow immunoassay. To solve this problem, the present invention takes advantage of a) the novel carbohydrate binding chemistry in the hydrogel particles, and b) the internal high surface area of the hydrogel particles within an internal open volume that is 95 percent solute. Horse Radish Peroxidase (HRP) commonly used for immunoassay amplification is a glycoprotein. We can bind HRP via the carbohydrate ligand in the hydrogel particles, permitting it to retain full enzymatic activity within the internal volume of the hydrogel particles. Use of the carbohydrate affinity bait will concentrate the HRP in the hydrogel particles to achieve a high internal concentration many orders of magnitude greater than simple volume exchange. The same hydrogel particle will capture the pathogen glycan analyte with high affinity. As shown in FIG. 1 this enables the creation of a lateral flow immunoassay in which an enzymatically amplified color reaction occurs inside the hydrogel particles containing the captured analyte only when the hydrogel particles are bound to the antibody detection line in a lateral flow format. The hydrogen peroxide cofactor for the HRP color substrate is generated at the capture line by solid phase glucose oxidase and glucose, a well-established method used in diabetes one step glucose monitoring. This color reaction occurs rapidly (30 seconds) because of the open volume of the hydrogel particles, and can increase visual detection sensitivity >1000 fold.

Addressing the urgent need for Improved point of care diagnosis of TB and Chagas Tuberculosis (TB) is one of the most important bacterial infections globally (9 million patients globally). WHO estimates that TB kills 1.8 million people yearly. The mortality rate for untreated TB is 68%, compared to 5% following treatment. Consequently, a reliable highly sensitive point-of care test for active pulmonary tuberculosis (TB) is critically important for disease control. Since urine can be easily collected non-invasively, it is an ideal biofluid to detect TB antigens. Unfortunately, TB urine testing has been hampered in the past because TB antigens exist in very low concentration, are masked by high abundant urinary resident proteins, and are subject to enzymatic degradation. Commercial tests that screen for the presence of mannose-capped lipoarabinomannan (LAM), a lipoglycan essential for the virulence of TB, lack adequate sensitivity for pulmonary TB, and cannot be used for HIV negative population screening (85% of TB patients) (13). Chagas disease is caused by Trypanosoma cruz; infection and is responsible for high mortality and morbidity among the world's poorest populations. 6 million to 7 million people worldwide are infected with the parasite. The disease is endemic in 21 countries of Latin America, where it causes more deaths than malaria, but can remain asymptomatic for many years. Chronic symptomatic disease, which can be fatal, develops in up to 30% of cases. Congenital transmission accounts for 25% of new infections with an estimate of 15,000 infected infants per year. The earlier in life congenital infection is detected, the higher the efficacy and tolerability of treatment. Current screening programs have very low sensitivity, high cost, and requires trained personnel. A sensitive, specific and field-friendly screening test is urgently needed for effective Chagas disease newborn screening.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 shows an exploded view of the cassette test device of the present invention.

FIG. 2 depicts the current amperometric sensor of the present invention as a traditional 2-prong electrode.

FIG. 3 shows results of a test to determine if the hydrogel particles capture and arrest at the capture antibody line against the ESAT-6.

FIG. 4 depicts a result of adding the chromogenic substrate DAB to the reactive solution of G and GOx, producing a dark brown precipitate and amplifies the colorimetric intensity of the LFI.

FIG. 5 depicts a paired t-test with unequal variance, showing results of the test and device of the present invention.

FIG. 6 exhibits a paired t-test with unequal variance was performed, and using the charge threshold, there was significant differentiation between the positive and negative test cases

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s). The invention is defined by the claims appended hereto.

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to effect such feature, structure, or characteristic in connection with other embodiments whether or not explicitly described.

The present invention is a test and device for Simultaneous Parallel Signal Amplification and Analyte-Ligand Capture functions configured to provide sufficient signal while additionally detecting low abundance biomarkers. The present invention employs a test, which, for convenience, is housed within a biological cartridge of a cassette. The biological cartridge of the present invention remains separated from an electronics section of the cassette, and only accommodates a nitrocellulose strip. The cassette of the present invention was inspired by current pregnancy tests available on the market. The cassette system of the test of the present invention allows for the biological cartridge to be removable and discarded after use. A new biological cartridge is inserted into the electronics section to start the next test, making the device of the present invention reusable.

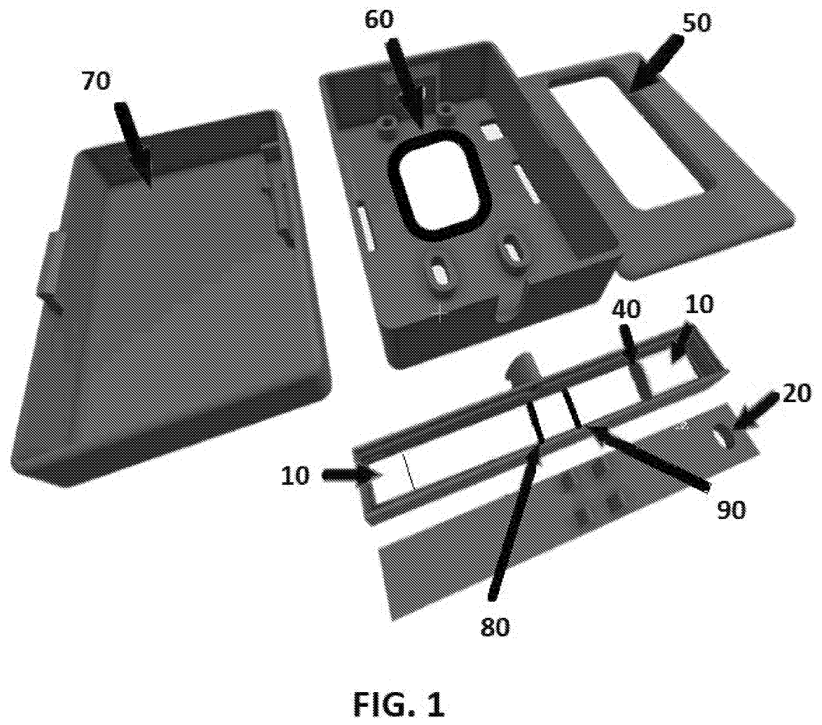

The cassette of the present invention is preferably printed with PLA material in its current state, which is very fast to prototype with. In the future, the model cassette is envisioned to be printed with a more resilient plastic such as ABS. It is also envisioned to be designed with a user-friendly design approachable for the end user. It should be understood that the current model depicted in FIG. 1 simply serves as a shell, and that future embodiments of the cassette may be more robust and/or have a differing form factor. The cassette, as shown disassembled/exploded in FIG. 1, includes an absorbent pad (10), an application site (20), an electrode site (30), a conjugate pad (40), a screen (50), a microcontroller (60), a battery (70), a test line (80), and a control line (90).

Capture Hydrogel Particles

Pertinent biomarkers found in blood and other bodily fluids are exceptionally low in concentration. Biomarkers are also subject to degeneration over time, especially during the processes of collecting and transporting samples.

Hydrogel particles are an open-mesh network of polymers with chemical affinity baits dyed to their surface, and can be used to overcome the obstacles discussed above. When combined with blood or bodily fluids, these hydrogel particles are able to segregate and eliminate significantly large amounts of undesirable proteins, enhance the target analytes, and amplify the sensitivity of immunoassays 100 fold. The chemical affinity baits have conductive elements within their structure, which can be exploited electrically. The manufacturing of the hydrogel particles is cost-effective, and the quality of this technology is able to maintain stability and complete solubility in bodily fluids over time. Additionally, these hydrogel particles can be functionalized with enzymes by affinity interactions. The hydrogel particles replace the traditional solid-core gold nanoparticles used in lateral flow immunoassays for the prototype of the present invention.

Immobilize and Detect: Amperometric Sensor

Enzymatic Amplification

Though the hydrogel articles contain ionic elements, it is imperative to amplify the signal in order to achieve sensitivity and performance standards. The method of amplification employed by the present invention produces both visual and electrical data. First, the hydrogel particles are functionalized with the enzyme horseradish peroxidase (HRP). HRP is an enzyme taken from the roots of horseradishes. Using enzymatic reactions to release electrons is a common and proven method of electrochemical sensing.

The system and device of the present invention combines the following chemicals and enzymes to increase the amperometric response: Glucose (G), Glucose Oxidase (GOx), Hydrogen Peroxide (H₂O₂), and HRP. GOx is an enzyme produced by fungi to readily oxidize G to produce H₂O₂. GOx is typically used to detect concentrations of G in medical diagnostics. In addition, it is an economical enzyme that has high durability.

The amplified signal will mainly be generated from the creation of H₂O₂ from G reducing GOx. After, HRP further reduces H₂O₂ to release electrons which interact with the sensor. The aforementioned reactions are simplified with the following equations:

D-Glucose+H₂O₂→D-Gluconic Acid+H₂O (1)

H₂O₂→HRP H2O+HRP (2)

To ensure that the amplification of the reactive solution is occurring, the chromogenic substrate DAB (3, 3-diaminobenzidine) is added to it. In the presence of the reactive solution, DAB produces a dark brown precipitate. Thus, the two-part amplification using enzymatic reactions and hydrogel particles with the DAB substrate will produce enhanced visual results as well as released electrons for electrochemical sensing.

Amperometric Sensor

Amperometric sensors are the most common and well-known electrochemical sensing devices, and will work best in conjunction with the enzymatic reaction described above. Amperometric sensors work in a framework of a voltage divider circuit, in which there is a variable resistor. The amperometric sensor behaves as the variable resistor. By applying a constant voltage potential across two electrodes, the change in current is detected. This sensing technique works best with recognizing oxidation and reduction reactions. In addition, amperometric sensing has compatibility with nanomaterials and has shown a wide range of nanotechnology applications to improve diagnostic sensing.

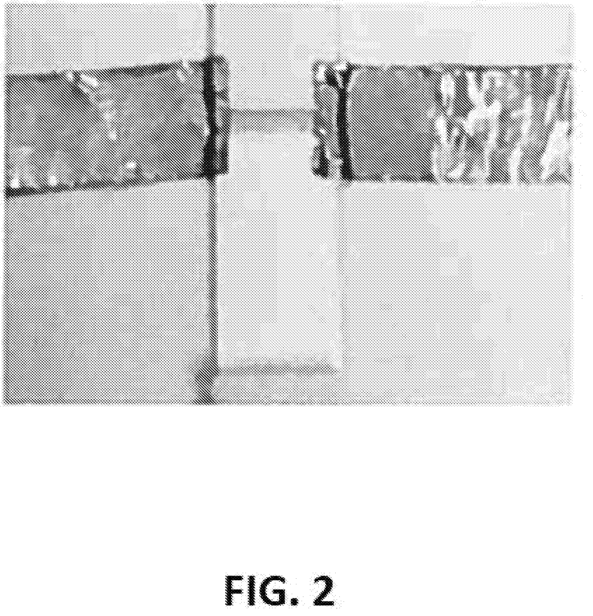

The electrical data acquisition system of the present invention consists of an Arduino pro mini, which is a low-cost microcontroller board, and an amperometric sensor across the test lines. The current amperometric sensor is a traditional 2-prong electrode as shown in FIG. 2. The Arduino samples the current across the test line at a frequency of 10 Hz. Since the enzymatic reaction takes place within seconds, it only samples the current for the first 15 seconds.

In theory, the hydrogel particles functionalized with HRP are immobilized at the antibody line in the positive test case will produce a higher current response in comparison to the negative test case, when the particles do not immobilize at the antibody line, and flow to the absorbent pad.

To further differentiate between the positive and negative test cases, the current is integrated over the 15 seconds period. This allows for a threshold to be established, allowing for a binary screening diagnostic of “LTBI Negative” or “LTBI Positive.” This threshold was found by running 15 positive and 15 negative tests, then examining the data for the decision boundary.

The final system of the present invention preferably functions as follows:
1. Analyte with TB antigen flows across the sample and conjugate pad.
2. The HRP/GOx functionalized hydrogel particles captures the TB antigen and continue to flow.
3. When the hydrogel particles reach the electrode system, the TB antigen binds with the TB capture antibody.
4. The G/GOx/H₂O₂/HRP reactions mentioned in equations 1, 2 occurs.
5. The free electrons generates a current response.
6. The functionalized hydrogel particles continue flowing across the electrode and are captured at the control line on the counter electrode.
7. The system gives a diagnostic read out based on the change in current on the working electrode.
   Overall, this approach combines the advantages of enzymatic reactions for amplifying a current response, the simplicity of lateral flow design, and the advanced protein capturing capabilities of the hydrogel particles for an optimal LTBI diagnostic test.

Dot Blots

To ensure that the reagents and antibodies used are compatible with each together, dot blot analyses were performed. A dot blot is a molecular biological assay that detects the presence of a target analyte. Since the hydrogel particles must be functionalized with HRP and they are targeting the LTBI antigen, ESAT-6, tests have been performed for the efficacy of the hydrogel particles being functionalized with HRP and capturing ESAT-6.

Efficacy of NP functionalization with HRP Enzyme

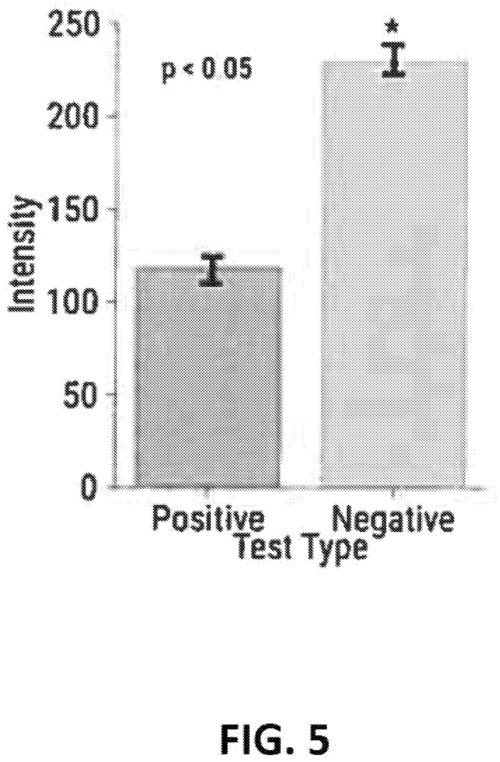

To test the efficacy of the hydrogel particles being functionalized with the HRP enzyme, washed hydrogel particles were incubated with HRP before proceeding with the standard dot blot procedure. FIG. 5 demonstrates that the particles can be functionalized with HRP when in PBS and urine. The RB-urine, AB-urine, and TB-urine spots are faint and do not show a dark signal like the initial solutions, functioning as the negative controls. The reactive black (RB) and alcian blue (AB) spots for urine and HRP were dark, similar to those of the initial solution, which indicate a positive result and thereby exhibit that the hydrogel particles were successfully functionalized with HRP.

Results

### NP Visual Amplification at Antibody Capture Line

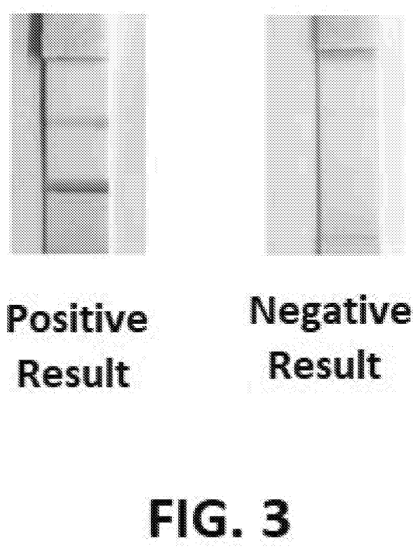

To demonstrate that the hydrogel particles are capturing the TB antigen, ESAT-6, and reactive solution is amplifying the visual response, a LFI was created using a conventional pregnancy test strip to determine if the hydrogel particles capture and arrest at the capture antibody line against the ESAT-6. Visual results are displayed in FIG. 3. The test strip with the positive test result demonstrates that the hydrogel particles have captured the TB biomarker, immobilized at the capture antibody line, and produced a dark brown precipitate. The negative test result demonstrates that the hydrogel particles produced no visually detectable line due to the absence of TB biomarkers.

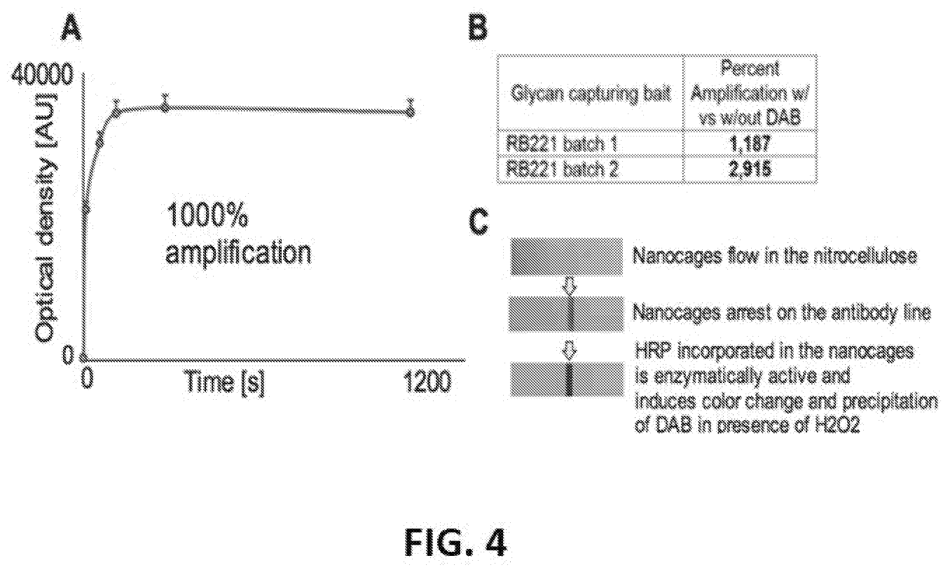

Thus, it has been shown that the hydrogel particles effectively capture ESAT-6 and arrest at the capture antibody line. Additionally, adding the reactive solution with the chromogenic substrate DAB will increase the visual response of the system 1000 fold within seconds, as exhibited in FIG. 3 and FIG. 4. FIG. 4 depicts adding the chromogenic substrate DAB to the reactive solution of G and GOx produces a dark brown precipitate within seconds and amplifies the colorimetric intensity of the LFI by 1000 fold.

FIG. 5 shows visual results of T-Test. A paired t-test with unequal variance is depicted, where p«0.05, n=50 samples, 25 positive, and 25 negative. Image Software was used to quantify the visual intensity of the lateral flow lines produced. Positive samples produce dark lines with low gray scale intensity values, while negative samples have whiter lines creating higher gray scale intensity values. This test was performed for 50 trials with 25 TB positive samples and 25 TB negative samples. To quantify the visual result generated at the test line, gray scale intensity software was employed. TB positive samples produced darker visual results generating a lower gray scale intensity measurement while TB negative samples did not produce a visual result, thus creating a higher gray scale intensity value as shown in FIG. 5.

To analyze the sensitivity of this visual amplification, a standard confusion matrix was used. Of the 25 known positive samples, the system of the present invention correctly identified them as TB positive, and out of the 25 known negative samples, the system of the present invention identified them as TB negative. This demonstrates that the visual amplification of the present invention achieves 100% sensitivity and 100% specificity over the 50 trials performed.

Amperometric results indicate that it is evident that, across the test line, there is a current for both positive and negative samples, as shown in FIG. 5. However, the current across the test line in the positive case is significantly higher, as it can be seen that it stabilizes around 50 μA while the negative test case is about 12 μA. This is due to the enzymatic reaction generating free electrons, as well as the conductive hydrogel particles between two prongs of the amperometric sensor, meanwhile the control does not have either of these two components.

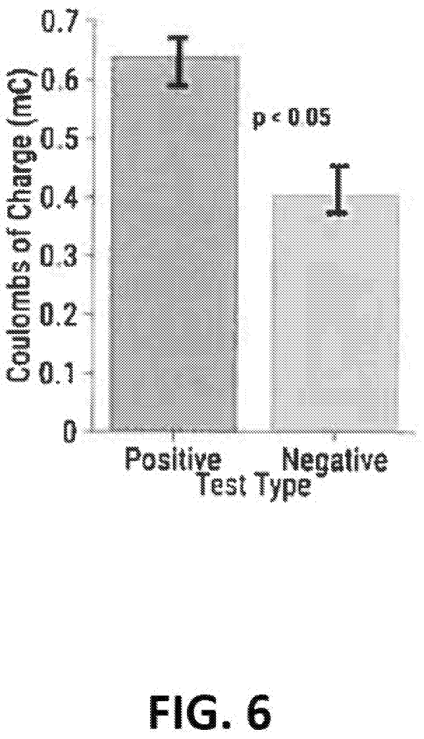

As shown in FIG. 6, a paired t-test with unequal variance was performed, and using the charge threshold, there was significant differentiation between the positive and negative test cases. Additionally, the average coulombs of charge transferred across the test line for the positive test case was averaged at 0.649 mC while the negative test case was averaged at 0.402 mC, demonstrating that the immobilization of the functionalized, copper-dyed particles produces a higher current at the test line.

Finally, a standard confusion matrix was used to assess the sensitivity and specificity of the electrical detection system. The electrical system correctly classified 24 of the 25 known positive test cases as TB positive, producing a 96% sensitivity. The system correctly classified 20 of the 25 known negative test cases at TB negative, producing an 80% specificity.

It should be understood that the purpose of the design process of the present invention was to develop a novel diagnostic that improved upon existing tools while also developing a quantifiable method of detection. This study has successfully demonstrated an improved method of capturing the ESAT-6 TB biomarker by utilizing hydrogel particles while also improving the sensitivity of a typical LFI. Combining the NP with enzymatic reactions and incorporating HRP within the volume of the hydrogel particles has enhanced the visual response of the LFI by 100%. Additionally, the enzymatic reactions have aided in the quantifiable detection of the hydrogel particles by using a biosensor. Amperometric methods of identifying free electrons has been successfully applied to this testing platform. Using simple sensor design, the system of the present invention has achieved 96% sensitivity and 80% specificity, thus providing a proof of concept that an amperiometric LFI using hydrogel particles for tuberculosis diagnosis (or other diagnoses) is feasible.

These results are valuable because the current TB diagnostics on the market fail to achieve the sensitivity and cost of the device of the present invention. Having a quantifiable LFI for TB, not only improves upon LFI sensitivity, but also produces data which could track the concentration of the target biomarker within a patient. Since the hydrogel particles can be configured for any biomarker, this methodology can be applied for a plethora of diseases and diagnostic applications. Additionally, having HRP within hydrogel particles is a completely new method of capturing and detecting the presence of low abundance biomarkers.

To improve this prototype, implementing AG/AgCl electrode designs, as well as incorporating a potentiostatic circuit design would greatly improve the variability seen in the electrical method of detection. Another suggestion for improvement could be incorporating new chromogenic substrates such as 3,3′, 5,5′-Tetramethylbenzidine or TMB. This substrate produces a blue color when exposed to a peroxidase reaction which would be beneficial to the test of the present invention since the hydrogel particle dye for tuberculosis is Reactive Blue 221, a blue color. Also, TMB has been known to produce better electrical effects, thus potentially enhancing the amperometric data.

The HRP incorporation inside the NP volume and attached to the dye bait can be modified. Currently, the system of the present invention has demonstrated that activated HRP retains its reactivity. However, this response could be greater if the addition of a spacer is added to provide space for the HPR to further catalyze or the HRP could be covalently bound to the hydrogel particles which would also promote better catalysis.

It is important to note that the visual and electrical systems can work independently, but in case the electrical components fail, the visual system functions on its own.

It should be understood that the hydrogel particles referenced are open porous hydrogel particles containing tunable pores and containing an immobilized novel high-affinity bait chemistry. The novel baits, which are derivatives of dyes, bind to proteins, nucleic acids, glycoproteins, glycolipids, and nucleic acids with extremely high affinity (10″2), preserving the captured analyte and effectively increasing the sensitivity of detection up to ten thousand fold. Hydrogel particles are commercialized, and have been applied to a wide variety of diagnostics and discovery projects by scientists worldwide. The bait chemistries of the present invention have provided a completely new approach to sequence the interface regions of interacting proteins. Such interfaces are the drug targets of the future. The system of the present invention engineers the bait of the hydrogel particles to create a slow release depot that can release therapeutic molecules in vivo.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

We claim:

1. A method for detecting analyte-ligand binding, comprising:
flowing an analyte with an antigen across a sample and conjugate pad; functionalized hydrogel particles capturing the antigen;
the functionalized hydrogel particles continuing to flow after capturing the antigen;
the antigen binding to a capture antibody when the functionalized hydrogel particles reach an electrode system;
reactions occurring;
free electrons generating a response;
the functionalized hydrogel particles continuing to flow across the electrode system; and
a control line on a counter electrode capturing the functionalized hydrogel particles.

2. The method of claim 1, wherein the reactions occurring are chosen from the group: D-Glucose+H₂O+O₂→D-Gluconic Acid+H₂O₂; H₂O₂+HRP_{red}→H₂O+HRPₒₓ

3. The method of claim 1, wherein the functionalized hydrogel particles have an enzyme placed in an internal volume of the functionalized hydrogel particles.

4. The method of claim 3, wherein the enzyme is horseradish peroxidase.

5. The method of claim 1, wherein the functionalized hydrogel particles have an enzymatic amplification system.

6. A system for detecting analyte-affinity molecule binding, comprising:
a particle;
an affinity molecule, configured to bind to an analyte of interest, within said particle; and an amplification reporter system associated with said particle.

7. The system of claim 6, wherein said particle is a porous polymer particle.

8. The system of claim 7, wherein said amplification reporter system is excluded from a surface of said particle.

9. The system of claim 8, wherein said particle has substantial open void volume.

10. The system of claim 9, wherein said amplification reporter system is an unlinked chemical reporter system.

11. The system of claim 9, wherein said amplification reporter system is an electrochemical reporter system.

12. The system of claim 9, wherein said affinity molecule is immobilized within an internal structure of said particle.

13. The system of claim 9, wherein a pore size of said particle is configured to allow a secondary affinity molecule to reach into an internal structure of said particle and to recognize said analyte bound to said affinity molecule.

14. The system of claim 13, wherein said secondary affinity molecule is chosen from the group: antibodies; antibody fragments; peptides; proteins; lipids; carbohydrates; nucleic acids, synthetic molecular recognition compounds.

15. The system of claim 13, wherein said pore size of said particle is configured to allow a secondary affinity molecule to reach into an internal structure of said particle and to recognize said analyte bound to said affinity molecule only when said particle resides at a predetermined detection zone.

16. The system of claim 15, wherein said amplification reporter system is configured to be activated after said secondary affinity molecule has recognized said analyte of interest at said predetermined detection zone.

17. The system of claim 16, wherein said amplification reporter system is configured to generate a signal signifying said particle in said pre-determined detection zone only when said analyte is present in said particle.

18. The system of claim 17, wherein said analyte is chosen from the group: metabolites; proteins; nucleic acids; lipids; hormones; cytokines; growth factors; biomarkers; virus particles; exosomes; bacteria; fungi; drug compounds; synthetic organic compounds; volatile odorants; toxicants; pollutants.

19. The system of claim 16, wherein said pre-determined detection zone has a sensor.
